(19) United States
(12) Patent Application Publication
(10) Pub. No.: US 2024/0350903 A1
(43) Pub. Date: Oct. 24, 2024

(54) CURRENT STATUS PRESENTATION SYSTEM, NON-TRANSITORY COMPUTER-READABLE RECORDING MEDIUM, AND CURRENT STATUS PRESENTATION METHOD

(71) Applicant: Anaguma Inc., Tokyo (JP)

(72) Inventor: Masaki Nishimura, Tokyo (JP)

(73) Assignee: Anaguma Inc., Tokyo (JP)

(21) Appl. No.: 18/763,522

(22) Filed: Jul. 3, 2024

Related U.S. Application Data
(63) Continuation of application No. PCT/JP2022/041987, filed on Nov. 10, 2022.

(30) Foreign Application Priority Data
Jan. 17, 2022 (JP) 2022-005024

(51) Int. Cl. A63F 13/216; A63F 13/211; A63F 13/87

(52) U.S. Cl. CPC A63F 13/216; A63F 13/211; A63F 13/87

(57) ABSTRACT

A current status presentation system includes a collector that collects, for each user who possesses a portable terminal, a plurality of types of information including position information, an acceleration, and operation management information indicating an operation status of an application software, a classifier that classifies, for each user, whether a current status of a user is moving or playing a game, based on a combination of an amount of change in the position information and a magnitude of the acceleration, the operation management information, and any one of classification algorithms, and a presenter that, when the current status is being moving, presents a first status image representing a classified current status in association with a user image, and when the current status is being playing the game, presents a second status image representing the classified current status in association with the user image.

### CROSS-REFERENCE TO RELATED APPLICATION

This application is a continuation application of International Application PCT/JP2022/041987 filed on Nov. 10, 2022 and designated the U.S., which claims the benefits of priorities of Japanese Patent Application No. 2022-005024 filed on Jan. 17, 2022, the entire contents of which are incorporated herein by reference.

### FIELD

The present disclosure relates to a current status presentation system, a non-transitory computer-readable recording medium, and a current status presentation method.

### BACKGROUND

There has been known a technology related to a messaging service for enabling a plurality of users to transmit and receive messages to and from respective terminals and to browse the messages (see, for example, Patent Document 1: Japanese Laid-open Patent Publication No. 2021-140231). In addition, there has been known a technology in which a sensor or the like acquires position information and acceleration of a terminal to estimate an action state of a user who possesses the terminal. The action state is an action related to the movement of the user in various actions that the user can take, such as a walking state, a running state, and a vehicle getting-on state (see, for example, Patent Document 2: International Publication Pamphlet No. WO2014/148077).

In addition, there has been known a technology in which sensor data is collected from the daily life of a user using a portable terminal equipped with various sensors (acceleration sensor, angular velocity sensor, heart rate sensor, and so on), and the user's behavior (while walking, running, riding a train, or the like) is identified (see, for example, Patent Document 3: Japanese Laid-open Patent Publication No. 2013-041323).

### SUMMARY

According to a first aspect of the present disclosure, there is provided a current status presentation system including: a collector configured to immediately collect, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed; a classifier configured to classify, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and a presenter configured to, when the current status is classified as being moving, immediately present a first status image representing a classified current status as an image of a moving body on a screen of the portable terminal in association with a user image representing the user with an image, and when the current status is classified as being playing the game, immediately present a second status image representing the classified current status and different from the first status image on the screen of the portable terminal in association with the user image.

In the above current status presentation system, the collector may collect, as one of the plurality of types of information, first information detected by a sensor that is other than the GPS sensor and the acceleration sensor and mounted on the portable terminal.

In the above current status presentation system, the collector may collect second information managed by software installed in the portable terminal as one of the plurality of types of information.

In the above current status presentation system, the collector may collect, as the plurality of types of information, both of first information detected by a sensor that is other than the GPS sensor and the acceleration sensor and mounted on the portable terminal and second information managed by software installed in the portable terminal.

In the above current status presentation system, the classifier may select one of the plurality of types of classification algorithms as a classification algorithm optimum for the combination, and classify the current status of the user for each user based on a selected optimum classification algorithm and the combination, and the optimum classification algorithm may be a classification algorithm with a highest accuracy for identifying the current status of the user.

In the above current status presentation system, the classifier may select two or more classification algorithms suitable for the combination from the plurality of types of classification algorithms, and classify the current status of the user for each user based on the selected classification algorithms and the combination, and the classification algorithms suitable for the combination may be two or more types of classification algorithms arranged in a descending order of accuracy for identifying the current status of the user.

In the current status presentation system, the plurality of types of classification algorithms may include discriminant analysis, multiple regression analysis, an analysis method based on quantification theory, and decision tree analysis.

In the above current status presentation system, the first status image and the second status image may be animation images, and the portable terminal may be moved by the user during the game.

According to a second aspect of the present disclosure, there is provided a non-transitory computer-readable recording medium having stored therein a program for causing a computer to execute a process. The process includes: immediately collecting, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed; classifying, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and when the current status is classified as being moving, immediately presenting a first status image representing a classified current status as an image of a moving body on a screen of the portable terminal in association with a user image representing the user with an image, and when the current status is classified as being playing the game, immediately presenting a second status image representing the classified current status and different from the first status image on the screen of the portable terminal in association with the user image.

According to a third aspect of the present disclosure, there is provided a current status presentation method executed by a computer to execute a process. The process includes: immediately collecting, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed; classifying, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and when the current status is classified as being moving, immediately presenting a first status image representing a classified current status as an image of a moving body on a screen of the portable terminal in association with a user image representing the user with an image, and when the current status is classified as being playing the game, immediately presenting a second status image representing the classified current status and different from the first status image on the screen of the portable terminal in association with the user image.

### BRIEF DESCRIPTION OF THE DRAWINGS

FIG. 1 is a diagram illustrating an example of a current status presentation system;

FIG. 2 is a diagram illustrating an example of a hardware configuration of the current status presentation system;

FIG. 3 is a diagram illustrating an example of a functional configuration of the current status presentation system;

FIG. 4 is a diagram illustrating an example of collected information;

FIG. 5 is a diagram illustrating an example of user information;

FIG. 6 is a diagram illustrating an example of image information;

FIG. 7 is a flowchart illustrating an example of the operation of the current status presentation system; and

FIG. 8 is a diagram illustrating an example of a screen of a portable terminal.

### DESCRIPTION OF EMBODIMENTS

However, with the above-described position information, acceleration, angular velocity, and heartbeat, there is a possibility that a user who transmits a message cannot accurately understand the current status of a transmission destination (i.e., a recipient) before starting communication, for example, before the transmission of the message. For example, a recipient may not be moving but is watching a video without moving in the same position, another recipient may be talking on a phone, and the other recipient may be sleeping. In such a case, the above-described technology cannot necessarily understand the current status of the recipient with high accuracy.

Accordingly, an object of one aspect of the present disclosure is to provide a current status presentation system, a non-transitory computer-readable recording medium, and a current status presentation method which present the current status of each user having a portable terminal with high accuracy.

Hereinafter, an embodiment of the present disclosure will be described with reference to the drawings.

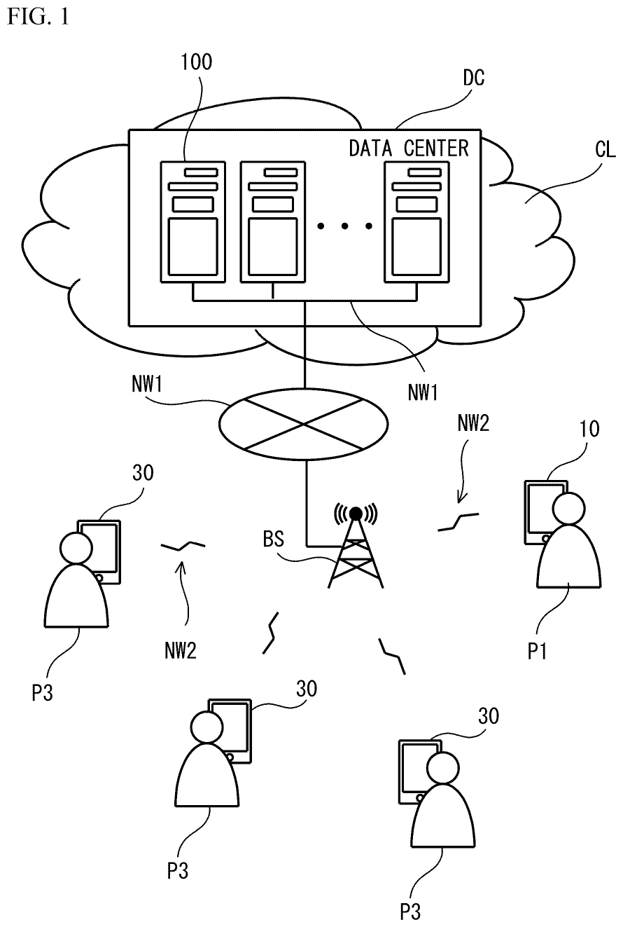

As illustrated in FIG. 1, a current status presentation system 100 is implemented in a data center DC on a cloud CL (specifically, a public cloud). In FIG. 1, the current status presentation system 100 is illustrated by one server, but various functions of the current status presentation system 100 may be distributed to a plurality of servers according to the functions, and may communicate with each other to cooperate with each other.

The current status presentation system 100 communicates with a portable terminal 10 possessed by a user P1 using the current status presentation system 100 and with portable terminals 30 possessed by a plurality of users P3 using the current status presentation system 100. More specifically, the current status presentation system 100 communicates with the portable terminals 10 and 30 via a wired communication network NW1, a portable base station BS, and a wireless communication network NW2. The wired communication network NW1 is a communication network such as an Internet or a LAN (Local Area Network). The wireless communication network NW2 is a communication network using, for example, LTE (Long Term Evolution).

For example, if the portable terminals 10 and 30 are included in a communicable region of the portable base station BS, the current status presentation system 100 communicates with the portable terminals 10 and 30 via the wired communication network NW1, the portable base station BS, and the wireless communication network NW2. A current status presentation application for communicating with the current status presentation system 100 is installed in the portable terminals 10 and 30. The current status presentation application is an application program for presenting the current statuses of the users P1 and P3 in cooperation with the current status presentation system 100. In FIG. 1, the smartphones are illustrated as examples of the portable terminals 10 and 30, but each of the portable terminals 10 and 30 may be a terminal having at least portability and a communication function, such as a tablet terminal, a smart watch, a VR (Virtual Reality) device, a game terminal, or a wearable device.

In this embodiment, the user P3 represents an acquaintance of the user P1. The acquaintances include, for example, friends, family, relatives, colleagues, superiors, subordinates, and the like, but are not particularly limited thereto as long as they have some kind of relationship with the user P1. When the user P1 operates the portable terminal 10 to start an application (specifically, application software) communicating with the current status presentation system 100, the current status presentation system 100 presents the current status of the user P3 to the portable terminal 10 in the form of, for example, an animation image.

Thereby, for example, before the user P1 transmits a message to the user P3, the user P1 can understand that the user P3 is currently watching a moving image or that the user P3 is sleeping. Therefore, even if the user P1 transmits the message to the user P3, the user P1 can immediately determine that there is no reply to the message, and thus the user P1 can be prevented from feeling irritated. Hereinafter, the current status presentation system 100 will be described in detail.

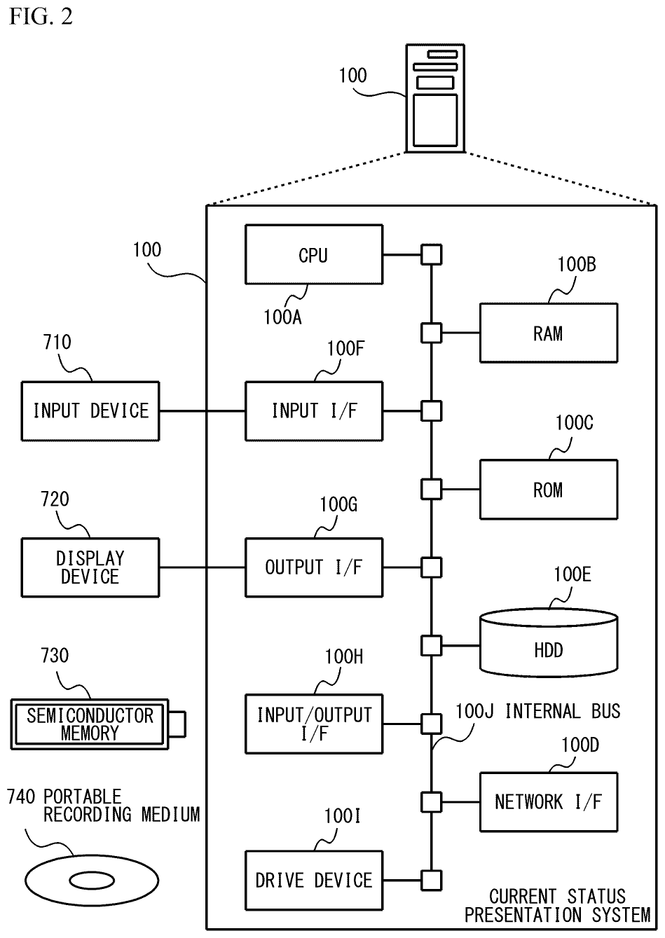

First, the hardware configuration of the current status presentation system 100 will be described with reference to FIG. 2. The hardware configuration of each of the portable terminals 10 and 30 is basically the same as that of the current status presentation system 100, but each of the portable terminals 10 and 30 further includes a plurality of sensors. The sensors include, for example, an acceleration sensor, a GPS (Global Positioning Systems) sensor, an image sensor, an illuminance sensor, a temperature and humidity sensor, an angular velocity sensor, a heart rate sensor, and the like.

As illustrated in FIG. 2, the current status presentation system 100 includes a CPU 100A as a processor, a RAM 100B and a ROM 100C as memories, and a network I/F (interface) 100D. The current status presentation system 100 may include at least one of a hard disk drive (HDD) 100E, an input I/F 100F, an output I/F 100G, an input/output I/F 100H, and a drive device 100I, as needed. The CPU 100A, the RAM 100B, the ROM 100C, the network I/F 100D, the HDD 100E, the input I/F 100F, the output I/F 100G, the input/output I/F 100H, and the drive device 100I are connected to each other by an internal bus 100J. A computer can be realized by cooperating at least the CPU 100A and the RAM 100B with each other.

An input device 710 is connected to the input I/F 100F. The input device 710 may be, for example, a keyboard or a mouse. The output I/F 100G is connected to a display device 720. The display device 720 may be, for example, a liquid crystal display. The input/output I/F 100H is connected to a semiconductor memory 730. The semiconductor memory 730 may be, for example, a USB (Universal Serial Bus) memory or a flash memory. The input/output I/F 100H reads a current status presentation program stored in the semiconductor memory 730. The input I/F 100F and the input/output I/F 100H include, for example, USB ports. The output I/F 100G includes, for example, a display port.

A portable recording medium 740 is inserted into the drive device 100I. As the portable recording medium 740, for example, a removable disk such as a CD (Compact Disc)-ROM or a DVD (Digital Versatile Disc) is available. The drive device 100I reads the current status presentation program recorded in the portable recording medium 740. The network I/F 100D includes, for example, a LAN port. The network I/F 100D is connected to the wired communication network NW1.

The CPU 100A stores the current status presentation program stored in the ROM 100C or the HDD 100E in the RAM 100B. The CPU 100A stores the current status presentation program recorded on the portable recording medium 740 in the RAM 100B. The CPU 100A executes the stored current status presentation program, whereby the current status presentation system 100 realizes various functions described later and executes various processes described later. The current status presentation program may be executed in accordance with a flowchart described later.

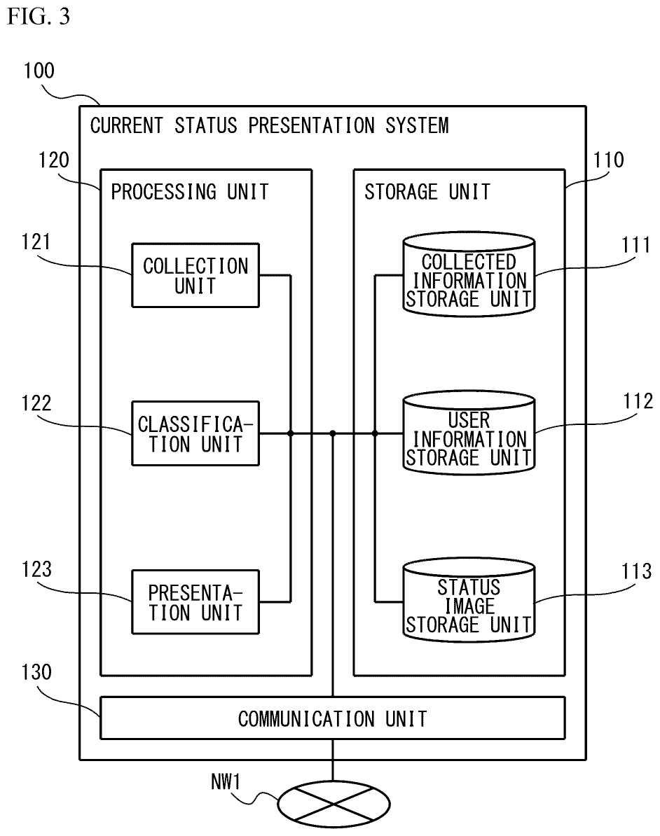

Next, the functional configuration of the current status presentation system 100 will be described with reference to FIG. 3. FIG. 3 illustrates a main part of the functions of the current status presentation system 100.

As illustrated in FIG. 3, the current status presentation system 100 includes a storage unit 110, a processing unit 120, and a communication unit 130. The storage unit 110 can be realized by one or both of the above-described RAM 100B and the above-described HDD 100E. The processing unit 120 can be realized by the above-described CPU 100A. The communication unit 130 can be realized by the above-described communication I/F 100D. Therefore, the storage unit 110, the processing unit 120, and the communication unit 130 are connected to each other. The storage unit 110 includes a collected information storage unit 111, a user information storage unit 112, and a status image storage unit 113. The processing unit 120 includes a collection unit 121, a classification unit 122, and a presentation unit 123.

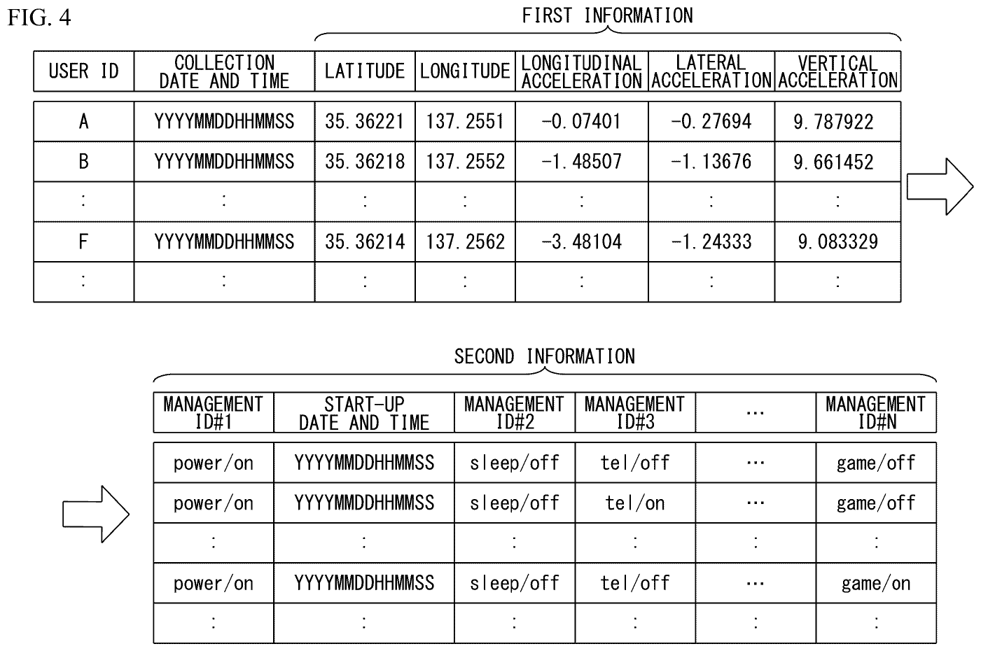

As illustrated in FIG. 4, the collected information storage unit 111 stores a plurality of types of retained information held by each of the portable terminals 10 and 30 as collected information. The collected information includes a user ID, a collection date and time, first information, and second information. The first information is information detected by sensors mounted on each of the portable terminals 10 and 30. The first information includes position information such as a latitude and a longitude detected by the GPS sensor, and a longitudinal acceleration, a lateral acceleration, and a vertical acceleration detected by the acceleration sensor. The first information may include a temperature and a humidity detected by the above-described temperature and humidity sensor.

The second information is information managed by the software installed in each of the portable terminals 10 and 30. The software may be an OS (Operating System), a battery management application, a video viewing application, a telephone application, a game application, or the like. The software may be the above-described current status presentation application. For example, if the power of each of the portable terminals 10 and 30 is ON, information “power/on” indicating that the power is ON is registered in a management ID #1 included as one of the items of the second information. If the current status presentation application installed in each of the portable terminals 10 and 30 is started, the start-up date and time of the current status presentation application is registered in the start-up date and time included as one of the items of the second information. By comparing the start-up date and time of the current status presenting application with the current date and time, it is possible to specify how many minutes ago, how many hours ago, or how many days ago the current status presenting application was started. The current date and time may be the system date and time managed by the current status presentation system.

If a sleep mode is not set in each of the portable terminals 10 and 30, information “sleep/off” indicating that the sleep mode is not set is registered in the management ID #2 included as one of the items of the second information. The sleep mode is a mode in which, for example, the screen display is stopped to suppress power consumption, and the reception of push notification, electronic mail, incoming call, and the like are waited. In addition, if the phone application or the game application is operating, information “tel/on” or “game/on” indicating that the application is operating is registered in the corresponding item. Accordingly, if the video viewing application is operating, information corresponding to the video viewing application is registered in the corresponding item. When the users P1 and P3 actively (or spontaneously) set their own statuses to the applications for presenting the current status independently from the sensors and the application other than the application for presenting the current status, information indicating the status is registered in the corresponding item. For example, when the user P1 sets a concentration mode indicating that the user P1 is concentrating on a specific or unspecified object (specifically, when the switch of the concentration mode is turned on), information corresponding to the concentration mode is registered in the corresponding item.

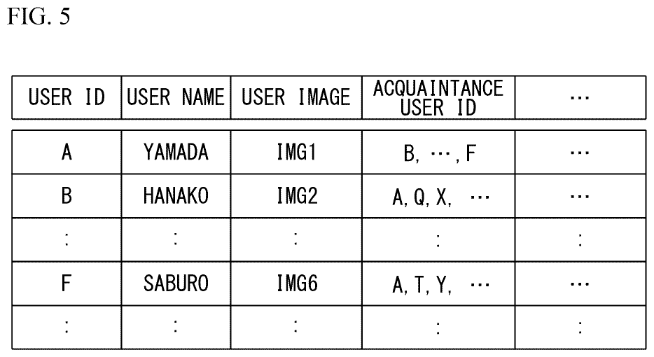

As illustrated in FIG. 5, the user information storage unit 112 stores information of the users P1 and P3 who have the portable terminals 10 and 30, respectively, in which the current status presentation applications are installed, as user information. The user information is requested when the current status presentation application is downloaded to each of the portable terminals 10 and 30, and is stored in the user information storage unit 112. The user information includes a user ID, a user name, a user image, an acquaintance user ID, and the like. Other items may be included in the user information as appropriate.

The user ID includes an identifier for identifying each of the users P1 and P3. The user name includes the name of each of the users P1 and P3. The user name may be registered with a surname alone or a name alone. The user image includes an identification image representing each of the users P1 and P3. The identification image may be, for example, an image of a face photograph of each of the users P1 and P3, or an image of a portrait created by each of the users P1 and P3. The identification image may be an image selected by each of the users P1 and P3 from among images of a plurality of characters prepared in advance by the current status presentation system 100. The acquaintance user ID is a user ID of a user who has an acquaintance relationship with the user P1 or P3 identified by the user ID. For example, it is possible to identify that the user P1 identified by the user ID “A” has the acquaintance relationship with the user P3 identified by the user ID “B”, and the user P3 identified by the user ID “F”.

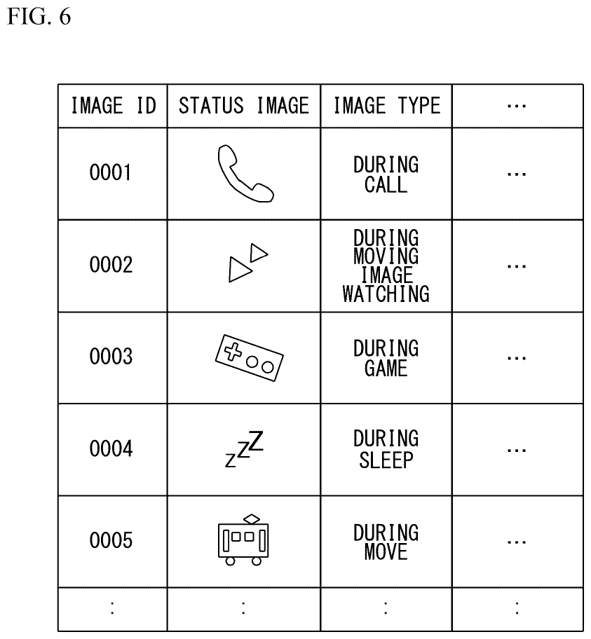

As illustrated in FIG. 6, the status image storage unit 113 stores an image representing the current status of each of the users P1 and P3 as image information. The image information includes an image ID, a status image, an image type, and the like. The image information may include items other than these items. The image ID is an identifier for identifying the status image. The status image is an animation image representing the current status of each of the users P1 and P3. In FIG. 6, the status image is illustrated as a still image, but if it is an image of a handset identified by the image ID “0001”, for example, it may tremble in small increments or change in size continuously or in stages. The operation of the image of the handset may be changed based on a predetermined operation setting. The images identified by the remaining image IDs are also changed in an animated manner based on the previous operation settings. The image type indicates the meaning of the status image.

The collection unit 121 collects the plurality of types of retained information held by each of the portable terminals 10 and 30 immediately (i.e., in real time) for each of the users P1 and P3 holding the portable terminals 10 and 30. The collection of the retained information may be periodic collection every several seconds or every several minutes. The plurality of types of retained information collected by the collection unit 121 include the above-described first information and the above-described second information. When the collection unit 121 collects the retained information, the collected retained information is stored as the collected information in the collected information storage unit 111. Thus, the collected information storage unit 111 stores the collected information.

The classification unit 122 accesses the collected information storage unit 111 to acquire the collected information. When the classification unit 122 acquires the collected information, the classification unit 122 classifies the current statues of the users P1 and P3 for each of the users P1 and P3, based on a combination of several types of information in the collected information for each of the users P1 and P3, and any one of a plurality of types of classification algorithms that classify the current statuses of the users P1 and P3. The plurality of types of classification algorithms include discrimination analysis, multiple regression analysis, decision tree analysis, an analysis method based on a quantification theory, and the like, and are implemented in the classification unit 122. A classification algorithm other than these classification algorithms may be implemented in the classification unit 122. Examples of the analysis method based on the quantification theory include quantification type 1, quantification type 2, and quantification type 3. While the multiple regression analysis, the discriminant analysis, and the decision tree analysis are performed on numerical data, the analysis method based on the quantification theory is performed on categorical data (classified data) obtained by appropriately dividing and categorizing quantitative data.

For example, if the longitudinal acceleration, the lateral acceleration, and the vertical acceleration are all equal to or more than the predetermined accelerations, the user P3 may be playing a game with much motion of the user P3 or the user P3 may be moving. On the other hand, if the longitudinal acceleration, the lateral acceleration, and the vertical acceleration are all less than the predetermined accelerations, there is a high possibility that the user P3 is in a state of calling, watching a moving image, or sleeping with little motion. In this way, the classification unit 122 can roughly or provisionally classify the current status of the user P3 according to the longitudinal acceleration, the lateral acceleration, and the vertical acceleration.

Here, for example, if the information “game/on” indicating the operation of the game application is registered in the management ID #N together with the longitudinal acceleration, the lateral acceleration, and the vertical acceleration having the predetermined accelerations or more, the classification unit 122 can uniquely identify that the user P3 is playing the game as the current status of the user P3. On the other hand, if the information “game/off” indicating the pause of the game application is registered in the management ID #N or if the amount of change in latitude and longitude is large, the classification unit 122 can uniquely identify that the user P3 is moving as the current status of the user P3. In this way, the classification unit 122 can uniquely identify the current status of the user P3 by a combination of a plurality of types of information.

Furthermore, the accuracy of identifying the current status of the user P3 may differ depending on the combination of the plurality of types of information and the plurality of types of classification algorithms. For example, as a plurality of types of information that can classify a state during sleeping, a first combination of information is assumed that the longitudinal acceleration, the lateral acceleration, and the vertical acceleration all indicate 0 (zero) and information “sleep/on” indicating that the sleep mode is set is registered in the management ID #2. In addition to such a combination, as the plurality of types of information that can classify the state during sleeping, a second combination of is also assumed that, for example, information “power/off” indicating that the power is not turned on is registered in the management ID #1, and the current time managed by the current status presentation system 100 indicates a midnight band.

In such a case, the accuracy of identifying the current status of the user P3 differs depending on the classification algorithms applied to the first combination or the second combination of information. For example, the classification unit 122 applies the discriminant analysis as a classification algorithm to the first combination of information, and the classification unit 122 applies the regression analysis or the decision tree analysis as a classification algorithm to the second combination of information. In this case, there is a high possibility that the result of applying the discriminant analysis to the first combination is different from the result of applying the regression analysis to the second combination. That is, there is a high possibility that the accuracies of identifying the current status of the user P3 differs from each other. For example, if the discriminant analysis is applied to the first combination of information, the current status of the user P3 may be classified as sleep. On the other hand, if the regression analysis is applied to the second combination of information, the current status of the user P3 may be classified as the operation business of an overnight bus or overnight train, rather than the sleep.

Accordingly, the classification unit 122 selects one of a plurality of types of classification algorithms as an optimum classification algorithm for the combination of the information, and classifies the current status of the user P3 based on the selected optimum classification algorithm and the combination of the information. The optimal classification algorithm is a classification algorithm with the highest accuracy for identifying the current status of the user P3. The classification unit 122 calculates a plurality of results by individually applying a plurality of types of classification algorithms to various combinations of information, and identifies a combination of several types of information and the classification algorithm which represent the highest value of the results from the plurality of results. The classification unit 122 determines the classification algorithm of the identified combination as the optimum classification algorithm. This makes it possible to classify the current status of the user P3 with high accuracy. The classification unit 122 executes such processing for each user ID of the users P1 and P3. As a result, for example, the current status of the user P3 can be classified with high accuracy as sleeping, or the current status of another user P3 can be classified with high accuracy as moving. The current status of the user P1 can be classified with high accuracy in the same manner. For example, if information corresponding to the concentration mode is registered in the corresponding item of the second information in association with the user ID “A” of the user P1, and if the longitudinal acceleration, the lateral acceleration, and the vertical acceleration are all equal to or more than the predetermined accelerations and the position information continuously changes, it is possible to classify that the user P1 is concentrated due to the operation business of the overnight bus or the overnight train. In this case, even if the user P1 is moving, the fact that the user P1 is in the concentration mode may be classified with priority given to the image type “moving”.

When two or more combinations of the several types of information and the classification algorithm which represent the highest value are identified, the classification unit 122 may select two or more types of classification algorithms suitable for the combination of the information from the plurality of types of classification algorithms, and classify the current status of the users P1 and P3 for each user based on the selected combination of the classification algorithm and the information. The classification algorithm suitable for the combination of information is two or more types of the classification algorithms which are arranged in descending order of accuracy for identifying the current statuses of the users P1 and P3. That is, the classification unit 122 may individually apply two or more types of classification algorithms to combinations of information, calculate a plurality of results representing average values of the results, and identify the combination of two or more types of classification algorithms and several types of information which represent the highest value of the results from the plurality of results.

The presentation unit 123 instantly presents status images representing the current statuses of the users P1 and P3 classified by the classification unit 122 on the screens of the portable terminals 10 and 30 in association with the user images, respectively. Specifically, when the presentation unit 123 acquires character strings representing the current statuses of the users P1 and P3 classified by the classification unit 122 for each user ID, the presentation unit 123 accesses the status image storage unit 113 and extracts status images corresponding to the acquired character strings. The presentation unit 123 accesses the user information storage unit 112 and extracts user images corresponding to the user IDs. The presentation unit 123 transmits the extracted status images to the portable terminals 10 and 30 via the communication unit 130 in association with the extracted user images, respectively. Thereby, the status images are displayed on the screens of the portable terminals 10 and 30 in association with the user images of the users P1 and P3, which will be described in detail later. Therefore, for example, the user P1 can quickly understand the current status of the user P3 who is an acquaintance of the user P1, with high accuracy.

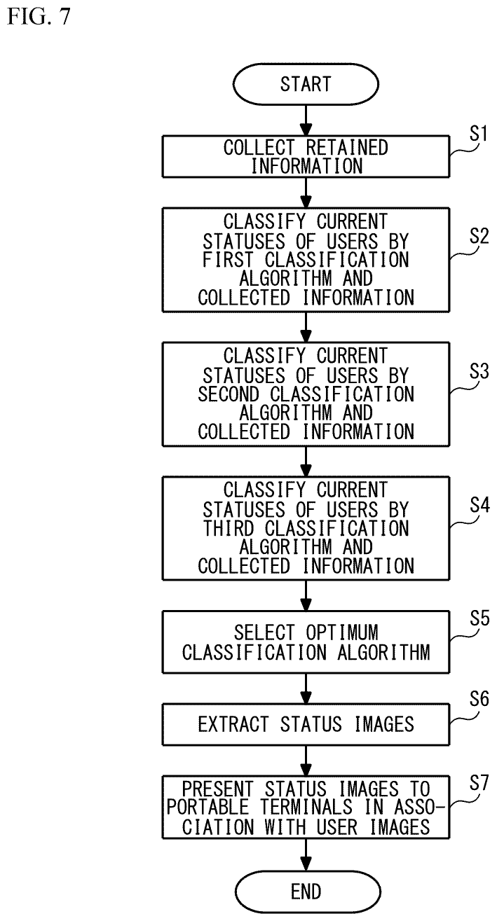

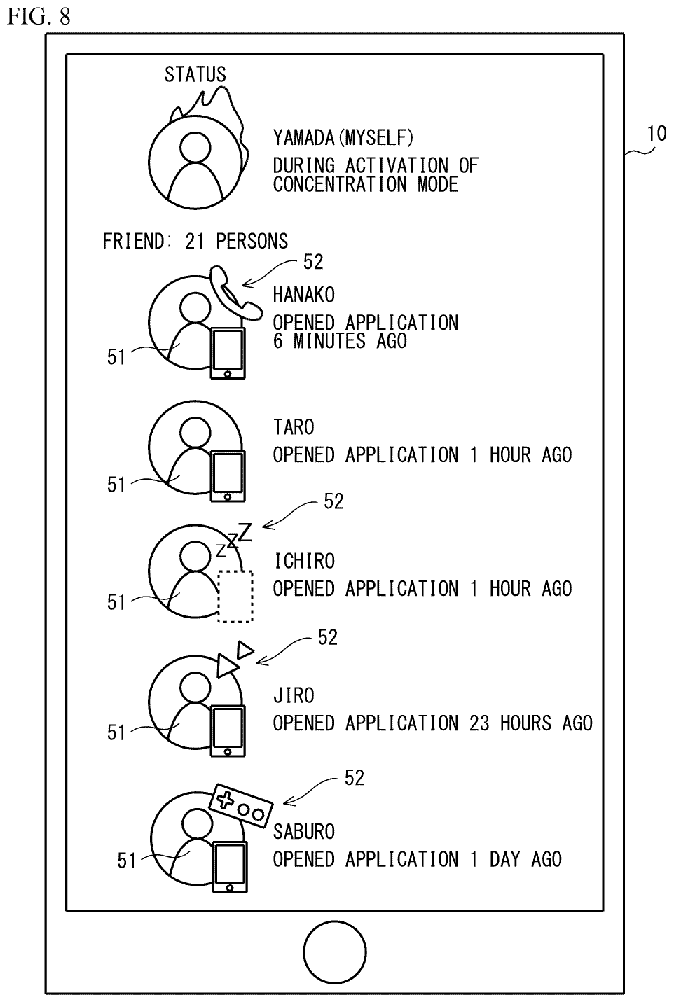

Next, the operation of the current status presentation system 100 will be described with reference to FIGS. 7 and 8.

First, as illustrated in FIG. 7, the collection unit 121 collects the retained information held by the portable terminals 10 and 30 (step S1). When the collection unit 121 collects the retained information, the collection unit 121 stores the collected retained information as the collected information in the collected information storage unit 111. When the collection unit 121 stores the collected information, the classification unit 122 classifies the current statuses of the users P1 and P3 by the first classification algorithm and the collected information in each unit of the user IDs (step S2). For example, the discriminant analysis can be adopted for the first classification algorithm. Instead of the discriminant analysis, the regression analysis may be adopted for the first classification algorithm, or the decision tree analysis may be adopted. The classification unit 122 classifies the current status of the users P1 and P3 by the first classification algorithm and the several types of information included in the collected information in each unit of the user IDs. The several types of information to be applied to the first classification algorithm can be set in advance. The classification unit 122 may apply each combination of all types of information to the first classification algorithm to classify the current statuses of the users P1 and P3, and select the current status of the result representing the highest value from among the plurality of classified results.

When the process of step S2 is completed, the classification unit 122 classifies the current statuses of the users P1 and P3 by the second classification algorithm and the collected information in each unit of the user IDs (step S3). The regression analysis may be adopted for the second classification algorithm, for example. Instead of the regression analysis, the decision tree analysis may be adopted for the second classification algorithm. The classification unit 122 classifies the current statuses of the users P1 and P3 by the second classification algorithm and the several types of information included in the collected information. The several types of information to be applied to the second classification algorithm can also be set in advance. The classification unit 122 may apply the combination of all types of information to the second classification algorithm to classify the current statuses of the users P1 and P3, and select the current status of the result representing the highest value from among the plurality of classified results.

When the process of step S3 is completed, the classification unit 122 classifies the current statuses of the users P1 and P3 by the third classification algorithm and the collected information in each unit of the user IDs (step S4). The decision tree analysis may be adopted for the third classification algorithm, for example. The classification unit 122 classifies the current statuses of the users P1 and P3 by the third classification algorithm and the several types of information included in the collected information. The several types of information to be applied to the third classification algorithm can also be set in advance. The classification unit 122 may apply the combination of all types of information to the third classification algorithm to classify the current statuses of the users P1 and P3, and select the current status of the result representing the highest value from among the plurality of classified results.

When the process of step S4 is completed, the classification unit 122 selects an optimum classification algorithm from the first classification algorithm, the second classification algorithm, and the third classification algorithm in each unit of the user IDs (step S5). For example, the classification unit 122 compares the result of the classification by the first algorithm, the result of the classification by the second algorithm, and the result of the classification by the third algorithm, identifies a combination of the several types of information and the classification algorithm which represent the highest value of the results from among the plurality of results, and selects the classification algorithm of the identified combination as the optimum classification algorithm. The classification unit 122 identifies the character strings representing the current statuses of the users P1 and P3 classified by the optimum classification algorithm in each unit of the user IDs.

When the process of step S5 is completed, the presentation unit 123 extracts status images (step S6). More specifically, the presentation unit 123 extracts status images corresponding to the character strings identified by the classification unit 122, and also extracts user images corresponding to the user IDs. When the process of step S6 is completed, the presentation unit 123 presents the status images to the portable terminals 10 and 30 in association with the user images, respectively (step S7).

Thereby, as illustrated in FIG. 8, status images 52 associated with user images 51 are displayed for each of the users P1 and P3 on the screen of the portable terminal 10, for example. In this way, the current status presentation system 100 can present the current status of each of the users who possess the portable terminals 10 and 30 with high accuracy. Thus, for example, the user P1 can understand the current status of the user P3 before starting communication with the user P3 (for example, before sending a message or starting a call) with high accuracy. In particular, since the status image 52 is displayed as an animation image, the user P1 can visually understand the current status of the user P3 in an instant.

If the sleep mode is set to the mobile terminal 30 or the power of the mobile terminal 30 is not turned on, the display may be made to be distinguishable by the presence or absence of coloring from another mobile terminal 30 in which the sleep mode is not set and the power is tuned on. Thereby, as illustrated in FIG. 8, the user P1 can easily distinguish the image representing the portable terminal 30 possessed by the user P3 having the user name “Ichiro” from the image representing the another portable terminal 30 possessed by the user P3 other than the user name “Ichiro”, and can instantly make a decision to exclude the image of the user name “Ichiro” from the communication partner.

Although the embodiments of the present disclosure have been described in detail, it is to be understood that the various change, substitutions, and alterations could be made hereto without departing from the spirit and scope of the invention. For example, the collected information may include a character string appearing in a social networking service (SNS), and the current status of the user may be classified into a state of being in a meal (more specifically, during breakfast, dinner, or the like), a state of being in a class, or a state of being on the way to school based on the character string. Further, it may be classified that the user P3 is climbing a mountain or on the sea based on the latitude, the longitude, the temperature, and the humidity.

For example, in the above-described embodiment, a physical server is used as an example of the current status presentation system 100, but the current status presentation system 100 may be a virtual server. Furthermore, the functions of the current status presentation system 100 may be distributed to a plurality of servers in accordance with a load or a type of service, or the respective storage units may be distributed to a plurality of storage units in accordance with a load or a management aspect. Further, the classification algorithm may be a learned model generated by collecting the retained information before the current status presentation method is performed and performing machine learning using the collected retained information as teacher data.

What is claimed is:

1. A current status presentation system comprising:
a collector configured to immediately collect, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed;
a classifier configured to classify, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and
a presenter configured to, when the current status is classified as being moving, immediately present a first status image representing a classified current status as an image of a moving body on a screen of the portable terminal in association with a user image representing the user with an image, and when the current status is classified as being playing the game, immediately present a second status image representing the classified current status and different from the first status image on the screen of the portable terminal in association with the user image.

2. The current status presentation system according to claim 1, wherein
the collector collects, as one of the plurality of types of information, first information detected by a sensor that is other than the GPS sensor and the acceleration sensor and mounted on the portable terminal.

3. The current status presentation system according to claim 1, wherein
the collector collects second information managed by software installed in the portable terminal as one of the plurality of types of information.

4. The current status presentation system according to claim 1, wherein
the collector collects, as the plurality of types of information, both of first information detected by a sensor that is other than the GPS sensor and the acceleration sensor and mounted on the portable terminal and second information managed by software installed in the portable terminal.

5. The current status presentation system according to claim 1, wherein
the classifier selects one of the plurality of types of classification algorithms as a classification algorithm optimum for the combination, and classifies the current status of the user for each user based on a selected optimum classification algorithm and the combination, and
the optimum classification algorithm is a classification algorithm with a highest accuracy for identifying the current status of the user.

6. The current status presentation system according to claim 1, wherein
the classifier selects two or more classification algorithms suitable for the combination from the plurality of types of classification algorithms, and classifies the current status of the user for each user based on the selected classification algorithms and the combination, and
the classification algorithms suitable for the combination are two or more types of classification algorithms arranged in a descending order of accuracy for identifying the current status of the user.

7. The current status presentation system according to claim 1, wherein
the plurality of types of classification algorithms include discriminant analysis, multiple regression analysis, an analysis method based on quantification theory, and decision tree analysis.

8. The current status presentation system according to claim 1, wherein
the first status image and the second status image are animation images, and
the portable terminal is moved by the user during the game.

9. A non-transitory computer-readable recording medium having stored therein a program for causing a computer to execute a process, the process comprising:
immediately collecting, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed;
classifying, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and
when the current status is classified as being moving, immediately presenting a first status image representing a classified current status as an image of a moving body on a screen of the portable terminal in association with a user image representing the user with an image, and when the current status is classified as being playing the game, immediately presenting a second status image representing the classified current status and different from the first status image on the screen of the portable terminal in association with the user image.

10. A current status presentation method executed by a computer to execute a process, the process comprising:
immediately collecting, for each user who possesses a portable terminal, a plurality of types of information including position information by a GPS (Global Positioning Systems) sensor, an acceleration by an acceleration sensor, and operation management information indicating an operation status of an application software, the portable terminal being equipped with the GPS sensor and the acceleration sensor and having game application software installed;
classifying, for each user, whether a current status of a user is moving or playing a game, based on a combination of an amount of change in the position information and a magnitude of the acceleration included in the plurality of types of information for each user, the operation management information, and any one of a plurality of types of classification algorithms for classifying the current status of the user; and
when the current status is classified as being moving, immediately presenting a first status image representing a classified current status as an image of a moving body on a screen of the portable terminal in association with a user image representing the user with an image, and when the current status is classified as being playing the game, immediately presenting a second status image representing the classified current status and different from the first status image on the screen of the portable terminal in association with the user image.
